(19) United States
(12) Patent Application Publication
(10) Pub. No.: US 2023/0040993 A1
(43) Pub. Date: Feb. 9, 2023

(54) A FIBER-OPTIC WAVE GUIDE SENSOR OF APTAMERS AND A DETECTION METHOD OF ITS APPLICATION

(71) Applicant: CAPITAL NORMAL UNIVERSITY, Beijing (CN)

(72) Inventors: Xinhui LOU, Beijing (CN); Jiaxing ZHAO, Beijing (CN); Zhangwei LU, Beijing (CN); Shuo WANG, Beijing (CN)

(21) Appl. No.: 17/618,084

(22) PCT Filed: Mar. 16, 2020
(86) PCT No.: PCT/CN2020/079442

§ 371 Date: Dec. 10, 2021

(30) Foreign Application Priority Data
Jun. 13, 2019 (CN) 201910509959.0

(51) Int. Cl. G01N 33/533; C12N 15/115

(52) U.S. Cl. CPC G01N 33/533; C12N 15/115

(57) ABSTRACT

The invention relates to a fiber-optic wave guide sensor of aptamers having functions of in situ target enrichment and purification, and a method for detection of small molecules to realize the quantitative detection of small molecules targets based on that small molecules targets and the aptamers complementary short strand DNA competitively bind with aptamers tethered on the fiber surface. It synchronously realized specifically binding aptamers with targets and in situ target enrichment and purification of targets by modifying aptamers and solid micro extraction layer with silica fibers of the fiber-optic wave guide sensor, which can achieve the ultrasensitive and ultrahigh specific quick detection for all types of small molecule targets regardless of any signal amplification reaction based on enzyme. The detection limitation is very low with good generalizability.

### PRIORITY CLAIM

This is a U.S. national stage of application No. PCT/CN2020/079442, filed on Mar. 16, 2020. Priority is claimed on the following application: Country: China, Application No.: 201910509959.0, Filed: Jun. 13, 2019, the entire content of which is incorporated here by reference.

### REFERENCE TO SEQUENCE LISTING SUBMITTED VIA EFS-WEB

This application includes an electronically submitted sequence listing in .txt format. The .txt file contains a sequence listing entitled “SWIP-002PUS_ST25.txt” created on Oct. 11, 2022 and is 3,474 bytes in size. The sequence listing contained in this .txt file is part of the specification and is hereby incorporated by reference herein in its entirety.

### TECHNICAL FIELD

The invention relates to a fiber-optic wave guide sensor of aptamers, and more particularly, to a fiber-optic wave guide sensor of aptamers having functions of in situ target enrichment and purification (SPME-OWS), which can achieve the ultrasensitive and ultrahigh specific quick detection for all types of small molecule targets regardless of water solubility, belonging to analytical chemistry technical field.

### BACKGROUND OF THE DISCLOSURE

Small organic molecules are one large classification of food and environmental contaminants, having features of numerous varieties, different water solubility, and less specific antibody, etc. Therefore, their analytical test mainly use the ways based on the Large-scale Instruments, such as Vapour phase chromatography, High performance liquid chromatography (HPLC), Gas chromatography mass spectrometry, liquid chromatography mass spectrometry, etc., which require expensive equipment, high work environment, and equipment maintenance, and which are not suitable for field testing. In recent years, various biosensors are developing at flying speed for satisfying quick detection for small molecule targets.

Aptamers are the single-stranded or the double-stranded DNA or RNA obtained by Systematic Evolution of Ligands by Exponential Enrichment (SELEX) technology (Nature, 1990, 346, 818-822; Nature, 1992, 355, 564-566). Aptamers can specifically recognize the varied target molecule ranging from proteins, small molecules, cells, to tissue, which have high chemical stability, are easy to synthetize and modify, have low cost, and have broad application prospects in biosensor field. Aptamers for detection of small molecules especially are very attractive as the high specificity antibodies of small molecules are not easy to be obtained. However, affinity of aptamers of small molecules is generally much lower than antibody. In general, signal is enlarged based on enzyme and Nanomaterial for improving detection sensitivity, but detection sensitivity cannot achieve the actual required level.

The fiber-optic wave guide sensor is one kind of portable fluorescent sensor. It is mainly based on that, total reflection of light is generated when laser enters into the light sparing substance from the light dense substance at a certain angle of incidence, a portion of laser will transmit in vertical direction of optical fiber, and the strength of this portion of laser will decrease exponentially with distance left from the optical fiber, which is called the evanescent wave. The evanescent wave can excite fluorescent group within the range of the evanescent wave transmission (the evanescent wave field). Therefore, target's antibody, receptor, and complementary chains of aptamers can be specifically recognized, or target molecules are attached on the surface of the optical fiber and fluorescence is labeled on target molecules, aptamers or antibody so as to realize quantitative fluorescence detection of target. The fiber-optic wave guide sensor is very suitable for cheap detection of environmental and food contaminants because it is simply and quickly operated, industrialization has been realized, regeneration of hundred times can be made on the sensing interface. However, the present detection limit is mostly the level of nM, which cannot achieve the limit standard of small molecules contaminants in the complex medium.

Extraction technology is one common sample preparation method in the analysis method based on the instrument, so as to remove substrate and enrich target to realize quantitative or qualitative detection of target. Solid-phase microextraction (SPME) is one new type of extraction technology in rapid development in recent years, which uses various enrichment materials attached to solid-phase to enrich and purify all kinds of targets (Trac-Trends in Analytical Chemistry 2018, 108, 154-166. Trac-Trends in Analytical Chemistry 2019. 110, 66-80).

### SUMMARY OF THE INVENTION

In order to overcome defection in the prior art, we combine SPME and aptamers for the first time in the invention, via simultaneous target enrichment, purification, and detection taking the fiber-optic wave guide sensor as an example so as to achieve the ultrasensitive and high specific detection for small molecule targets. The invention realized quantitative detection of small molecules in various complex substrates samples and all the detection limits are lower over 20 times than the State Limit Standard. Further, liquid samples only need to be diluted and solid samples only need to be extracted without the complicated sample pretreatment. The methods of the invention have enormous development prospects.

The object of the invention is to provide a fiber-optic wave guide sensor of aptamers having functions of in situ target enrichment and purification (SPME-OWS) and a detection method of its application to achieve the high sensitive and high specific detection for small molecule targets. The methods of the invention synchronously assembly the extraction layer SPME having high efficiency target extraction capability, for example, bare fiber, Tween 80, and aptamers having target specificity on the fiber-optic sensing interface to realize synchronous target enrichment, purification and the specific detection, which can achieve the ultrasensitive and ultrahigh specific detection. The invention can realize the quantitative detection of small molecules targets based on that small molecules targets and the aptamers complementary short strand DNA (cDNA) competitively bind with aptamers tethered on the fiber surface. The SPME on the fiber surface high effectively enriched small molecules in the solution nearby the fiber surface, which substantially bind small molecules with aptamers tethered on the fiber surface, substantially decreasing hybridization of the fluorophore labeled aptamers complementary DNA (cDNA) with aptamers, enabling the ultrasensitive and highly specific detection of targets. The method of the invention is a general way, taking detections of four representative environmental and food small molecules contaminants as examples, respectively comprising kanamycin A (Kana) of hydrophilic small molecule antibiotic, sulfadimethoxine (SDM) of hydrophobic small molecule antibiotic, Alternariol (AOH) of small molecule mycotoxin, and Di-(2-ethylhexyl) phthalate (DEHP) of high hydrophobic small molecule. Especially, the method of the invention can be used for the direct detection of targets in the complicated samples (milk, lake water, Wine, wheat), only requiring diluting the liquid sample, without any time consuming and complicated sample pretreatment. All detection sensitivity satisfies the limit standard of each target in the food and environment.

The method has the following advantages:

1) The method of the invention realizes synchronous target enrichment, purification and the specific detection, which is realized in the prior art for the first time. And the operation is very convenient and fast.

2) The method of the invention can achieve the ultrasensitive and ultrahigh specific detection, the detection of limits of which is lower 625-2000, 000 times than that of the conventional fiber-optic wave guide sensor, even still lower 325-20, 000 times than that of the electrochemical detection method.

3) The method of the invention has ultrahigh specificity (the selectivity>1000) and anti-matrix interference ability, which can be used for the direct detection of targets in the complicated samples (milk, lake water, Wine, wheat), only requiring to dilute the liquid sample, without any time consuming and complicated sample pretreatment. All detection sensitivity satisfies the limit standard of each target in the food and environment.

4) The sensor of the invention has superior target generality, which can used for high hydrophobic, hydrophobic and hydrophobic small molecules.

5) One of unique advantages of the invention is conveniently controlling of the detection sensitivity and dynamic interval. For example, the efficiency of in situ target enrichment and purification can be controlled by simply changing constitution of SPME or adding other ingredients which affects target enrichment in the buffers, so as to control the dynamic range. The detection sensitivity and dynamic interval of the sensor are adjusted by changing the surface density of the probe or using aptamers with the different affinity commonly in the prior art. However, it is very difficult for the accurate control of the probe density on the surface without good repeatability. Further, aptamers probes with the different affinity require the complicated engineering design. The method of the invention is more concise without these limitations.

6) The test cost is lower, and the stability is better among batches by using aptamers to realize the specific recognition of targets compared to the sensor based on antibody.

7) The sensor of the invention can recycle and reproduce for many times (>100 times) and has stability (Fluorescence signal change is ±6%).

8) The sensor of the invention can achieve quick detection within several minutes.

9) The sensor of the invention does not limit to be used for the small molecules, which can be used for other types of targets, for example, protein, heavy metal ion, etc., by changing extractant.

The specific experimental procedures of the invention are as follows:

1) Hydroxylation of the optic fiber surface: Firstly, The optical fiber with clean surface was dipped into a 3:1 v/v concentrated sulfuric acid and a 30% hydrogen peroxide mixing solution at 100-120° C. for 1 h, then, the fiber was taken from the mixing solution and washed to neutral with the ultrapure water, followed by blowing dry with nitrogen and drying in an oven at 70-90° C. for 4-6 h, taking the fiber in the dryer and cooling to room temperature;

2) Silylanization of the optic fiber surface: the above fiber was immersed APTS anhydrous toluene solution at room temperature for 1-2 h, followed by rinsing with Anhydrous toluene, toluene-ethyl alcohol (v/v=1:1) and ethyl alcohol wash (three time), blowing dry with nitrogen and drying in an oven at 180° C. for 4-6 h, taking the fiber in the dryer and cooling to room temperature;

3) Aptamers coupling of the optic fiber surface: the optical fiber of silylanization was immersed in 10 mM phosphate buffered solution (PB) containing glutaraldehyde for 4 h at room temperature, after the reaction being finished, washed with the ultrapure water three times, blowing dry with nitrogen. The fiber was then immersed in the amino modified aptamers solution 6-8 h at room temperature, washed then with the ultrapure water three times;

4) Restoring and sealing: the above fiber was immersed in sodium borohydride (NaBH₄) solution for 30 minutes, sealing the fiber interface with a certain concentration of extractant, for example, Tween 80 solution (When fabricating SPME-OWS of the bare fiber, the fiber interface was sealed without the extractant), washed then with the ultrapure water three times and stored in the refrigerator of 4° C.

5) The optic fiber was assembled into the reaction chamber of the waveguide sensor, after the baseline being stabled, pumping the mixed solution containing a certain concentration of small molecule target and complementary chains of fluorescent modified aptamers in the reaction chamber, measuring the change of fluorescence signal in real time;

6) The fiber was flushed with solution of sodium dodecyl sulfate (SDS) to regenerate the sensor interface; repeating 5);

7) Drawing the working curves of optical waveguide sensor detecting different targets;

8) Selectivity test: targets of 5) was changed to substances of Selectivity test.

### BRIEF DESCRIPTION OF THE DRAWINGS

FIG. 1 is a view of interfacial modification of the optic wave guide fiber (Part A), principle of evanescent wave optical excitation (Part B) and composition system of the optic wave guide sensor (Part C) in the invention.

FIG. 2 is a view of fabrication, detection and interface regeneration process of a fiber-optic wave guide sensor of aptamers having functions of in situ target enrichment and purification (SPME-OWS) in the invention.

FIG. 3 is a view of fabrication and detection process of OWS for the small molecule detection (classic-OWS) in the prior art.

FIGS. 4A and 4B are views of the working curves of detecting Kana (FIG. 4A) and SDM (FIG. 4B) in the buffer.

FIGS. 5A-5C represent fabricated SPME-OWS of the invention realizing the ultrasensitive and ultrahigh specific quick detection for Kana of hydrophilic small molecule. FIG. 5A is the working curves of detecting Kana in the buffer, FIG. 5B is the bar chart of selectivity tests for other small molecules (TET: tetracycline; AMP: ampicillin; SDM; DEHP), and FIG. 5C is the working curves of detecting Kana in the lake water and milk. All tests used buffer 1 (phosphate, 10 mM; NaCl, 50 mM; KCl, 5 mM; MgCl 5 mM; pH 7.0).

FIGS. 6A-6C represent fabricated SPME-OWS of the invention realizing the ultrasensitive and ultrahigh specific quick detection for SDM of hydrophobic small molecule. FIG. 6A is the working curves of detecting SDM in the buffer, FIG. 6B is the bar chart of selectivity tests for other small molecules (TET: tetracycline; AMP: ampicillin; SDM; DEHP), and FIG. 6C is the working curves of detecting SDM in the lake water and milk. All tests used buffer 2 (trihydroxymethylaminomethane, 20 mM; NaCl, 50 mM; KCl, 5 mM; MgCl 5 mM; pH 7.0).

FIGS. 7A-7C represent fabricated SPME-OWS sealed with Tween 80 of the invention realizing the ultrasensitive and ultrahigh specific quick detection for DEHP of high hydrophobic small molecule. FIG. 7A is the working curves of detecting DEHP in the buffer, FIG. 7B is the bar chart of selectivity tests for other small molecules and metal ion (SDM, Kana, Phthalic acid (BA), benzoic acid (PA), Hg2+, Pb2+), and FIG. 7C is the working curves of detecting DEHP in the lake water and Wine. All tests used buffer 3 (NaCl, 100 mM; trihydroxymethylaminomethane, 20 Mm; MgCl 2 mM; KCl, 5 mM; CaCl, 1 mM; 0.03% Triton X-100, 2% Dimethyl Sulfoxide, pH 7.9).

FIGS. 8A-8C represent fabricated SPME-OWS sealed with Tween 80 of the invention realizing the ultrasensitive and ultrahigh specific quick detection for Alternariol (AOH) of small molecule mycotoxin. FIG. 8A is the working curves of detecting AOH in the buffer, FIG. 8B is the bar chart of selectivity tests for other toxin small molecules AME, Patulin, ZEA, OTA, DON), and FIG. 8C is the working curves of detecting AOH in the Wheat Extract. All tests used buffer 4 (CaCl, 0.9 mM; KCl, 2.685 mM; KDP 1.47 mM; MgCl 0.49 mM; NaCl, 137 mM; DSP, 8.1 mM; pH 7.4).

FIGS. 9A and 9B represent fabricated SPME-OWS of the invention realizing convenient control of the detection dynamic range. FIG. 9A represents SPME-OWS realizing control of the detection dynamic range for SDM using the different buffers (buffer 1 and buffer 3). FIG. 9B represents SPME-OWS realizing control of the detection dynamic range for SDM using the different SPME layers (No sealing, Tween 80, BSA).

FIG. 10 represents fluorescent signals changes of multiple times' interface regeneration in the fiber surface of fabricated SPME-OWS sealed with Tween 80 of the invention.

### DETAILED DESCRIPTION OF PREFERRED EMBODIMENTS

The detailed descriptions of embodiments in the invention for further understanding of the invention are as follows. The following embodiments are used to illustrate the invention but are not used to limit the scope.

In general, the technical scheme of the invention can realize the quantitative detection of small molecules targets based on that small molecules targets and the aptamers complementary short strand DNA (cDNA) competitively bind with aptamers tethered on the fiber surface. The SPME on the fiber surface high effectively enriched small molecules in the solution nearby the fiber surface, which substantially bind small molecules with aptamers tethered on the fiber surface, substantially decreasing hybridation of the fluorophore labeled aptamers complementary DNA (cDNA) with aptamers, enabling the ultrasensitive and highly specific detection of targets. It comprises the following detailed experiment procedure:

1) Hydroxylation of the optic fiber surface: Firstly, The optical fiber with clean surface was dipped into a 3:1 v/v concentrated sulfuric acid and a 30% hydrogen peroxide mixing solution at 100-120° C. for 1 h, then, the fiber was taken from the mixing solution and washed to neutral with the ultrapure water, followed by blowing dry with nitrogen and drying in an oven at 70-90° C. for 4-6 h, taking the fiber in the dryer and cooling to room temperature;

2) Silylanization of the optic fiber surface: the above fiber was immersed APTS anhydrous toluene solution at room temperature for 1-2 h, followed by rinsing with Anhydrous toluene, toluene-ethyl alcohol (v/v=1:1) and ethyl alcohol wash (three time), blowing dry with nitrogen and drying in an oven at 180° C. for 4-6 h, taking the fiber in the dryer and cooling to room temperature;

3) Aptamers coupling of the optic fiber surface: the optical fiber of silylanization was immersed in 10 mM phosphate buffered solution (PB) containing glutaraldehyde for 4 h at room temperature, after the reaction being finished, washed with the ultrapure water three times, blowing dry with nitrogen. The fiber was then immersed in the amino modified aptamers solution 6-8 h at room temperature, washed then with the ultrapure water three times;

4) Restoring and sealing: the above fiber was immersed in sodium borohydride (NaBH₄) solution for 30 minutes, sealing the fiber interface with a certain concentration of extractant, for example, Tween 80 solution (When fabricating SPME-OWS of the bare fiber, the fiber interface was sealed without the extractant), washed then with the ultrapure water three times and stored in the refrigerator of 4° C.

5) The optic fiber was assembled into the reaction chamber of the waveguide sensor, after the baseline being stabled, pumping the mixed solution containing a certain concentration of small molecule target and complementary chains of fluorescent modified aptamers in the reaction chamber, measuring the change of fluorescence signal in real time;

6) The fiber was flushed with solution of sodium dodecyl sulfate (SDS) to regenerate the sensor interface; repeating 5);

7) Drawing the working curves of optical waveguide sensor detecting different targets;

8) Selectivity test: targets of 5) was changed to substances of Selectivity test.

TABLE 1
DNAs used in this invention

| | SEQ | | |
|---|---|---|---|
| | ID | | |
| Name | NO | Sequences (5′- 3′) | Description |
| NH₂-Kana | 1 | NH₂-(EG)₁₈-TGGGGGTTGAGGCTAAG | Amino group-modified Kana aptamer |
| | | CCGAGTCACTAT | for fabrication of Kana NADL-FOEW |
| NH₂-SDM | 2 | NH₂-(EG)₁₈-GAGGGCAACGAGTG | Amino group-modified SDM aptamer |
| | | TTTATAGA | for fabrication of SDM NADL-FOEW |
| NH₂-PAE | 3 | NH₂-(EG)₁₈-CTTTCTGTCCTTCCGTCA | Amino group-modified PAE aptamer |
| | | CATCCCACGCATTCTCCACAT | for fabrication of DEHP |
| | | | NADL-FOEW |
| NH₂-HSA | 4 | NH₂-AAAAAAAAAAGTGCCGAAAT | Amino group-modified HSA aptamer |
| | | ACGGCAC | for fabrication of HSA NADL-FOEW |
| c-Kana-Cy 5.5 | 5 | ATAGTGACTCGG-Cy 5.5 | Fluorophore Cy 5.5-tabled probe |
| | | | complementary to Kana aptamer |
| c-SDM-Cy 5.5 | 6 | AAACACTCGTTGCC-Cy 5.5 | Fluorophore Cy 5.5-labled probe |
| | | | complementary to SDM aptamer |
| c-PAE-Cy 5.5 | 7 | GGATGTGACGGAAG-Cy 5.5 | Fluorophore Cy 5.5-labled probe |
| | | | complementary to PAE aptamer |
| c-HSA-Cy 5.5 | 8 | AGCTTATGCGTAGCCTCTAGTGATT | Fluorophore Cy 5.5-labled HSA |
| | | AACGCAG-Cy 5.5 | aptamer partially complementary |
| | | | to HSA aptamer NH₂-HSA |
| Cy 5.5-PAE | 9 | Cy 5.5-CTTTCTGTCCTTCCGTCACAT | Fluorophore Cy 5.5-labled PAE |
| | | CCCACGCATTCTCCACAT | aptamer |
| Cy 5.5-Kana | 10 | Cy 5.5-TGGGGGTTGAGGCTAAGCCG | Fluorophore Cy 5.5-labled Kana |
| | | AGTCACTAT | aptamer |
| Cy 5.5-SDM | 11 | Cy 5.5-GAGGGCAACGAGTGTTTATA | Fluorophore Cy 5.5-labled SDM |
| | | GA | aptamer |

(EG): CH₂CH₂O

### Embodiment 1. Principle, Optic Fiber Fabrication, Target Test and Sensor Interface Regeneration Process of a Fiber-Optic Wave Guide Sensor of Aptamers Having Functions of In Situ Target Enrichment and Purification (SPME-OWS)

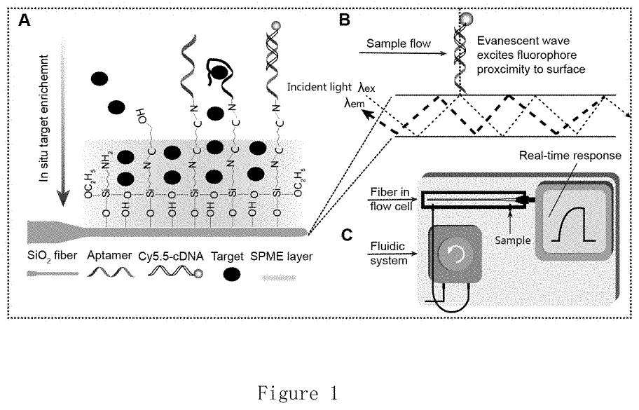

The invention provides a fiber-optic wave guide sensor of aptamers having functions of in situ target enrichment and purification (SPME-OWS) and a detection method of its application to achieve the high sensitive and high specific detection for small molecule targets. The principle of the invention is as shown in Part A of FIG. 1. Aptamers having target specificity assembled on the fiber-optic sensing interface to realize synchronous target enrichment, purification and the specific detection, which can achieve the ultrasensitive and ultrahigh specific detection. The invention can realize the quantitative detection of small molecules targets based on that small molecules targets and the aptamers complementary short strand DNA (cDNA) competitively bind with aptamers tethered on the fiber surface. The SPME layers (for example, nonenveloped fiber layer, Tween 80 absorbed layer) on the fiber surface high effectively enriched small molecules in the solution nearby the fiber surface, which substantially bind small molecules with aptamers tethered on the fiber surface, substantially decreasing hybridization of the fluorophore labeled aptamers complementary DNA (cDNA) with aptamers, enabling the ultrasensitive and highly specific detection of targets. As shown in Part B of FIG. 1, the fluorophore labeled subordinate DNA hybridized on the fiber surface was excited by the evanescent wave vertical to the fiber, the generated fluorescence emission was detected by the detector. The fluorescence intensity decreased as the increase of target concentration to realize the quantitative detection of target. As shown in Part C of FIG. 1, it is a view of composition system of the optic wave guide sensor in the invention, having small size, automatic operation with sample injection and data processing controlled by computer, convenient use.

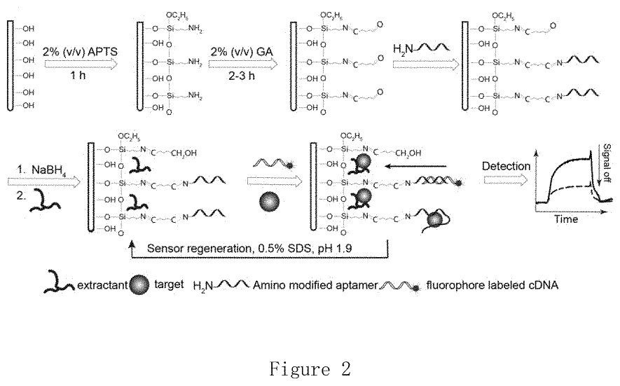

According to the invention, the fiber modification process of SPME-OWS is as shown in FIG. 2. Firstly, the optical fiber was immersed in 30% hydrofluoric acid (HF) to etch for 2-3 h till to have a diameter of about 220 μm with 3.5 cm insertion depth of optical fiber, washed to neutral with the ultrapure water, followed by in proper order: 1) Hydroxylation of the optic fiber surface, 2) Silylanization of the optic fiber surface, 3) Aptamers coupling of the optic fiber surface, 4) Restoring and sealing of the optic fiber surface (The sealing can be omitted according to target properties), and finishing fabrication process of the fiber. The detailed operation conditions are as follows.

1) Hydroxylation of the optic fiber surface: Firstly, The optical fiber with clean surface was dipped into a 3:1 v/v concentrated sulfuric acid and a 30% hydrogen peroxide mixing solution at 100-120° C. for 1 h, then, the fiber was taken from the mixing solution and washed to neutral with the ultrapure water, followed by blowing dry with nitrogen and drying in an oven at 70-90° C. for 4-6 h, taking the fiber in the dryer and cooling to room temperature;

2) Silylanization of the optic fiber surface: the above fiber was immersed APTS anhydrous toluene solution at room temperature for 1-2 h, followed by rinsing with Anhydrous toluene, toluene-ethyl alcohol (v/v=1:1) and ethyl alcohol wash (three time), blowing dry with nitrogen and drying in an oven at 180° C. for 4-6 h, taking the fiber in the dryer and cooling to room temperature;

3) Aptamers coupling of the optic fiber surface: the optical fiber of silylanization was immersed in 10 mM phosphate buffered solution (PB) containing glutaraldehyde for 4 h at room temperature, after the reaction being finished, washed with the ultrapure water three times, blowing dry with nitrogen. The fiber was then immersed in the amino modified aptamers solution 6-8 h at room temperature, washed then with the ultrapure water three times;

4) Restoring and sealing: the above fiber was immersed in sodium borohydride (NaBH₄) solution for 30 minutes, sealing the fiber interface with a certain concentration of extractant, for example, Tween 80 solution (When fabricating SPME-OWS of the bare fiber, the fiber interface was sealed without the extractant), washed then with the ultrapure water three times and stored in the refrigerator of 4° C.

The above fabricated optic fiber was assembled into the reaction chamber of the waveguide sensor to begin test of target. The fluorescent detector online installed on the sensor recorded changes of the fluorescent signals in real time for quantitative analysis of target concentration. After each test, washed the fiber with 0.5% SDS (pH=1.9) for 60 seconds to regenerate the sensing interface. After washed the fiber with the corresponding detection buffer again to begin the next test.

### Embodiment 2. Principle, Optic Fiber Fabrication, Target Test and Sensor Interface Regeneration Process of OWS (Classic-OWS) in the Prior Art

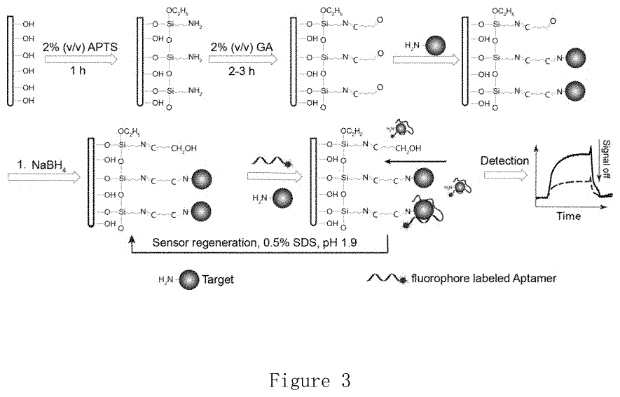

The fiber modification process of classic-OWS is as shown in FIG. 3. Firstly, the optical fiber was immersed in 30% hydrofluoric acid (HF) to etch for 2-3 h till to have a diameter of about 220 μm with 3.5 cm insertion depth of optical fiber, washed to neutral with the ultrapure water, followed by in proper order: 1) Hydroxylation of the optic fiber surface, 2) Silylanization of the optic fiber surface, 3) Kana A or SDM coupling of the optic fiber surface, 4) Restoring of the optic fiber surface, and finishing fabrication process of the fiber. The detailed operation conditions are as follows.

1) Hydroxylation of the optic fiber surface: Firstly, The optical fiber with clean surface was dipped into a 3:1 v/v concentrated sulfuric acid and a 30% hydrogen peroxide mixing solution at 100-120° C. for 1 h, then, the fiber was taken from the mixing solution and washed to neutral with the ultrapure water, followed by blowing dry with nitrogen and drying in an oven at 70-90° C. for 4-6 h, taking the fiber in the dryer and cooling to room temperature;

2) Silylanization of the optic fiber surface: the above fiber was immersed APTS anhydrous toluene solution at room temperature for 1-2 h, followed by rinsing with Anhydrous toluene, toluene-ethyl alcohol (v/v=1:1) and ethyl alcohol wash (three time), blowing dry with nitrogen and drying in an oven at 180° C. for 4-6 h, taking the fiber in the dryer and cooling to room temperature;

3) Kana Aor SDM coupling of the optic fiber surface: the optical fiber of silylanization was immersed in 10 mM phosphate buffered solution (PB) containing glutaraldehyde for 4 h at room temperature, after the reaction being finished, washed with the ultrapure water three times, blowing dry with nitrogen. The fiber was then immersed in the amino modified aptamers solution 6-8 h at room temperature, washed then with the ultrapure water three times;

4) Restoring: the above fiber was immersed in sodium borohydride (NaBH₄) solution for 30 minutes, washed then with the ultrapure water three times and stored in the refrigerator of 4° C.

The above fabricated optic fiber was assembled into the reaction chamber of the waveguide sensor to begin test of target. The fluorescent detector online installed on the sensor recorded changes of the fluorescent signals in real time for quantitative analysis of target concentration. After each test, washed the fiber with 0.5% SDS (pH=1.9) for 60 seconds to regenerate the sensing interface. After washed the fiber with the corresponding detection buffer again to begin the next test.

### Embodiment 3. Detection of Kana a and SDM in the Buffer Using OWS(Classic-OWS) of the Prior Art

Preparing Kana A and SDM standard solutions having the different final concentration (0, 10 pM, 100 pM, 1 nM, 10 nM, 100 nM, 200 nM, 500 nM, 800 nM, 1 μM, 10 μM) in buffer 2 (trihydroxymethylaminomethane, 20 mM; NaCl, 50 mM; KCl, 5 mM; MgCl 5 mM; pH 7.0), respectively mixing with 100 nM fluorescent modified aptamers (Cy5.5-Kana or Cy5.5-SDM, Table 1), in proper order putting in the optic-fiber sensor from the low concentration to the high concentration, after finishing each test, regenerating the interface and cleaning up the pipe to drop the fluorescent signal to the baseline, recording the fluorescent changes with time in the different concentrations, drawing the working curves with relative fluorescence signal reduction percentage value at different target concentrations being vertical coordinates.

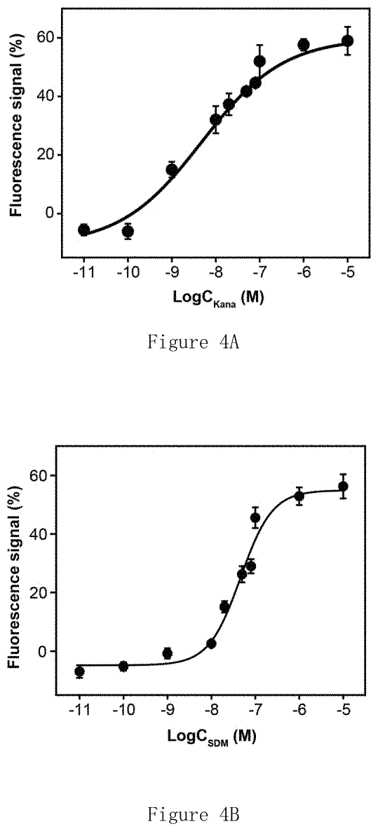

The result is as shown in FIGS. 4A-4B. The detection limitation is 0.5 nM for Kana A according to triple signal-to-noise ratio, and the detection dynamic range is 0.5 nM-10 μM (FIG. 4A); The detection limitation is 10.3 nM for SDM, and the detection dynamic range is, and the detection dynamic range is 10.3 nM-1 μM (FIG. 4B).

### Embodiment 4. The High Sensitive and High Specific Detection for Kana A in the Buffer, Lake Water and Milk Using SPMES-OWS (without Interface Sealing)

Hydroxylation, silylanization, coupling, sealing and restoring process of the optic fiber surface are same as Embodiment 1, without sealing after the fiber being restored, in which the coupling aptamer is NH2-Kana (Table 1). During experiment of the working curves of for Kana A, 0.5% SDS should be added to destroy the G-quadruplex structure formed by aptamer of Kana on the fiber surface.

Preparing Kana standard solutions having the different final concentration (0,100 fM, 1 pM, 10 pM, 100 pM, 1 nM, 10 nM, 100 nM, 1 μM, 10 μM) in buffer 1 ((phosphate, 10 Mm; NaCl, 50 mM; KCl, 5 mM; MgCl 5 mM; pH 7.0), respectively mixing with 100 nM fluorescent modified complementary chains of aptamers (Cy5.5-Kana or Cy5.5-SDM, Table 1), in proper order putting in the optic-fiber sensor from the low concentration to the high concentration, after finishing each test, regenerating the interface and cleaning up the pipe to drop the fluorescent signal to the baseline, recording the fluorescent changes with time in the different concentrations, drawing the working curves with relative fluorescence signal reduction percentage value at different target concentrations being vertical coordinates.

Respectively preparing TET, AMP, SDM and DEHP standard solutions having the final concentration of 10 nM to test target selectivity.

Respectively dissolving Kana (0 pM, 100 pM, 1 nM, 10 nM, 100 nM, 1 μM, 10 μM) in the lake water, using buffer 1 for 1000 times dilution, to detect Kana in the lake water. Respectively dissolving Kana (0 pM, 10 nM, 100 nM, 1 μM, 10 μM) in the skim milk, using buffer 1 for 10000 times dilution, to detect Kana in the milk.

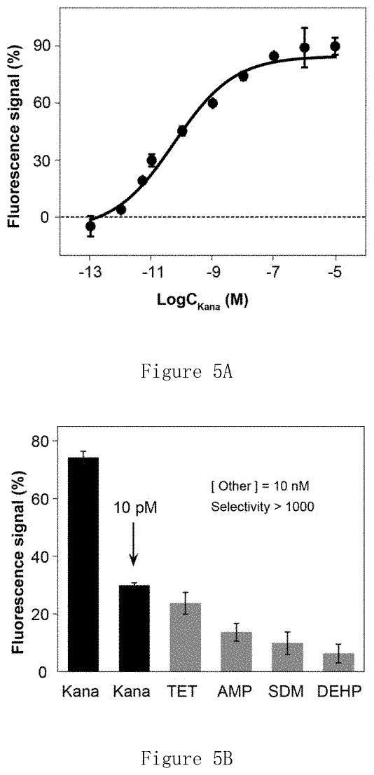

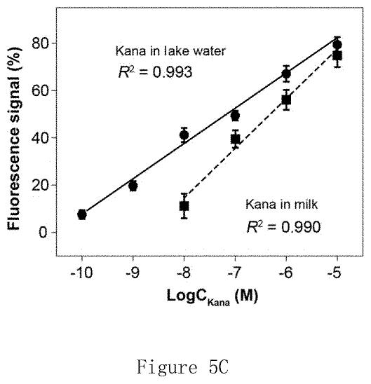

The result is as shown in FIG. 5A. The detection limitation is 800 fM, in which the sensitivity is less 625 times than the detection limitation of classic-OWS in the prior art, less 1250 times than the detection limitation of electrochemical sensors (Electrochimica Acta, 2015, 182, 516-523), and the dynamic range is 10 pM-100 nM. As shown in FIG. 5B, the fluorescent signal of 10 pM Kana A decreased much than that of 10 nM other small molecule and thus the target selectivity of the sensor is >1000. As shown in FIG. 5C, after the lake water sample containing Kana A was diluted for 1000 times, and detected directly without any sample pretreatment, the detection limitation is 100 pM, and the linear dynamic range is 100 pM-10 μM (R2=0.993). After the milk sample containing Kana A was diluted for 10000 times, and detected directly without any sample pretreatment, the detection limitation is 10 nM, and the linear dynamic range is 10 nM-10 μM (R2=0.990), in which the detection limitation is much less than 150 μg/L (257 nM) of the State standard limitation Kana A in the milk.

### Embodiment 5. The High Sensitive and High Specific Detection for SDM in the Buffer, Lake Water and Milk Using SPMES-OWS (without Interface Sealing)

Hydroxylation, silylanization, coupling, sealing and restoring process of the optic fiber surface are same as Embodiment 1, without sealing after the fiber being restored, in which the coupling aptamer is NH2-SDM (Table 1). Preparing SDM standard solutions having the different final concentration (0, 10 aM, 100 aM, 1 fM, 10 fM, 100 fM, 1 pM, 10 pM, 100 pM, 1 nM, 10 nM, 100 nM, 1 μM, 10 μM) in buffer 2 (trihydroxymethylaminomethane, 20 mM; NaCl, 50 mM; KCl, 5 mM; MgCl 5 mM; pH 7.0), respectively mixing with 100 nM fluorescent modified complementary chains of aptamers (c-SDM-Cy 5.5), in proper order putting in the optic-fiber sensor from the low concentration to the high concentration, after finishing each test, regenerating the interface and cleaning up the pipe to drop the fluorescent signal to the baseline, recording the fluorescent changes with time in the different concentrations, drawing the working curves with relative fluorescence signal reduction percentage value at different target concentrations being vertical coordinates.

Respectively preparing TET, AMP, SDM and DEHP standard solutions having the final concentration of 10 nM to test target selectivity.

Respectively dissolving SDM (0 pM, 1 pM, 10 pM, 100 pM, 1 nM, 10 nM, 100 nM, 1 μM, 10 μM) in the lake water, using buffer 2 for 1000 times dilution, to detect SDM in the lake water. Respectively dissolving SDM (0 pM, 3 nM, 30 nM, 50 nM, 300 nM, 3 μM, 5 μM) in the skim milk, using buffer 1 for 10000 times dilution, to detect SDM in the milk.

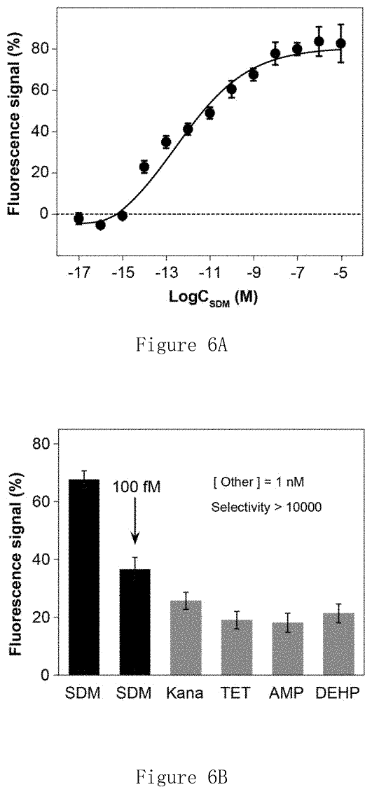

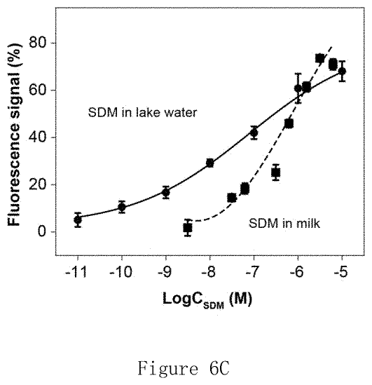

The result is as shown in FIG. 6A. The detection limitation is 4.8 fM, in which the sensitivity is less 2×106 times than the detection limitation of classic-OWS in the prior art, less 20000 times than the detection limitation of SDM electrochemical sensors reported by us (Sens. Actuators B 2017, 253, 1129-1136), and the dynamic range is 4.8 fM-10 nM. As shown in FIG. 6B, the fluorescent signal of 100 fM SDM decreased much than that of 1 nM other small molecule and thus the target selectivity of the sensor is >10000. As shown in FIG. 6C, after the lake water sample containing SDM was diluted for 1000 times, and detected directly without any sample pretreatment, the detection limitation is 10 pM, and the linear dynamic range is 10 pM-10 μM. After the milk sample containing SDM was diluted for 10000 times, and detected directly without any sample pretreatment, the detection limitation is 10 nM, and the linear dynamic range is 10 nM-1 μM, in which the detection limitation is much less than 2 mg/L (3.2 μM) of the State standard limitation of SDM in the milk.

### Embodiment 6. The High Sensitive and High Specific Detection for DEHP in the Buffer, Lake Water and Wine Using SPMES-OWS with Interface Sealing of Tween 80

Hydroxylation, silylanization, coupling, sealing and restoring process of the optic fiber surface are same as Embodiment 1, in which the coupling aptamer is NH2-DEHP (Table 1). Preparing DEHP standard solutions having the different final concentration (0, 1 fM, 10 fM, 100 fM, 1 pM, 10 pM, 100 pM, 1 nM, 10 nM, 100 nM, 200 nM) in buffer 3 (NaCl, 100 mM; trihydroxymethylaminomethane, 20 Mm; MgCl 2 mM; KCl, 5 mM; CaCl, 1 mM; 0.03% Triton X-100, 2% Dimethyl Sulfoxide, pH 7.9), respectively mixing with 100 nM fluorescent modified aptamers (c-DEHP-Cy5.5), in proper order putting in the optic-fiber sensor from the low concentration to the high concentration, after finishing each test, regenerating the interface and cleaning up the pipe to drop the fluorescent signal to the baseline, recording the fluorescent changes with time in the different concentrations, drawing the working curves with relative fluorescence signal reduction percentage value at different target concentrations being vertical coordinates.

Respectively preparing SDM, Kana, BA, PA, Hg2+, Pb2+ having the final concentration of 100 nM and fluorescent modified complementary chains of aptamers (c-DEHP-Cy 5.5) having the final concentration of 100 nM in buffer 3. Respectively mixing the every target solution with the complementary chain solution to test target selectivity according to the following procedure. The first step is putting buffer 3 into the reaction chamber for 60 seconds, the second step is putting the mixing solutions of target and complementary chain into the reaction chamber for 20 seconds and then retaining in the reaction chamber for 200 seconds, the third step is putting 0.5% SDS (pH 1.9) into the reaction chamber for 60 seconds, and finally putting buffer 3 into the reaction chamber for 50 seconds till the baseline returned to original position.

Respectively dissolving DEHP (0 pM, 100 pM, 1 nM, 10 nM, 100 nM) in the lake water, using buffer 3 for 1000 times dilution, to detect DEHP in the lake water. Respectively dissolving DEHP (0 pM, 1 nM, 5 nM, 10 nM, 50 nM, 100 nM) in the Wine, using buffer 3 for 1000 times dilution, to detect DEHP in the Wine.

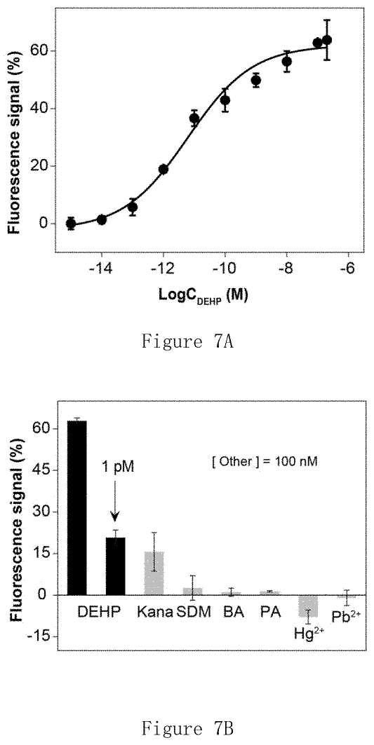

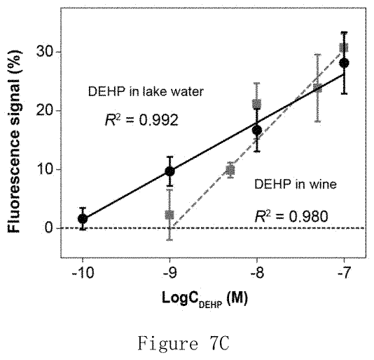

The result is as shown in FIG. 7A. The detection limitation is 40 fM, in which the sensitivity is less 350 times than the detection limitation of DEHP electrochemical sensors reported by us (Anal. Chem. 2017, 89, 5270-5277), and the dynamic range is 40 fM-100 nM. As shown in FIG. 7B, the fluorescent signal of 1 pM DEHP decreased much than that of 100 nM other small molecule and icon, thus the target selectivity of the sensor is >105. As shown in FIG. 7C, after the lake water sample containing DEHP was diluted for 1000 times, and detected directly without any sample pretreatment, the detection limitation is 100 pM, and the linear dynamic range is 100 pM-100 nM (R2=0.992). After the Wine sample containing DEHP was diluted for 1000 times, and detected directly without any sample pretreatment, the detection limitation is 1 nM, and the linear dynamic range is 1 nM-100 nM (R2=0.980), in which the detection limitation is much less than 0.3 μM of the State standard limitation of DEHP in the Wine.

### Embodiment 7. The High Sensitive and High Specific Detection for AOH in the Buffer and Wheat Using SPMES-OWS with Interface Sealing of Tween 80

Hydroxylation, silylanization, coupling, sealing and restoring process of the optic fiber surface are same as Embodiment 1, in which the coupling aptamer is NH2-AOH (Table 1). Preparing AOH standard solutions having the different final concentration (0, 10 fM, 100 fM, 1 pM, 10 pM, 100 pM, 1 nM, 10 nM, 100 nM, 1 μM) in buffer 4 (CaCl, 0.9 mM; KCl, 2.685 mM; KDP 1.47 mM; MgCl 0.49 mM; NaCl, 137 mM; DSP, 8.1 mM; pH 7.4), respectively mixing with 50 nM fluorescent modified aptamers (c-AOH-Cy 5.5, Table 1), in proper order putting in the optic-fiber sensor from the low concentration to the high concentration, after finishing each test, regenerating the interface and cleaning up the pipe to drop the fluorescent signal to the baseline, recording the fluorescent changes with time in the different concentrations, drawing the working curves with relative fluorescence signal reduction percentage value at different target concentrations being vertical coordinates.

Respectively preparing standard solutions of other toxin small molecules such as AME, Patulin, ZEA, OTA and DON having the final concentration of 100 pM to test target selectivity.

Respectively dissolving AOH (0, 100 fM, 1 pM, 10 pM, 100 pM, 1 nM, 10 nM, 100 nM) in the wheat extract (taking wheat flour, 1 g, adding pure acetonitrile, 6 mL, mediating the mixing solution for 3 minutes, then centrifugating for 10 minutes at 9000 r/min, taking the supernate, 1 mL, no membrane), using buffer 4 for 100 times dilution, to labelly detect AOH in the wheat extract.

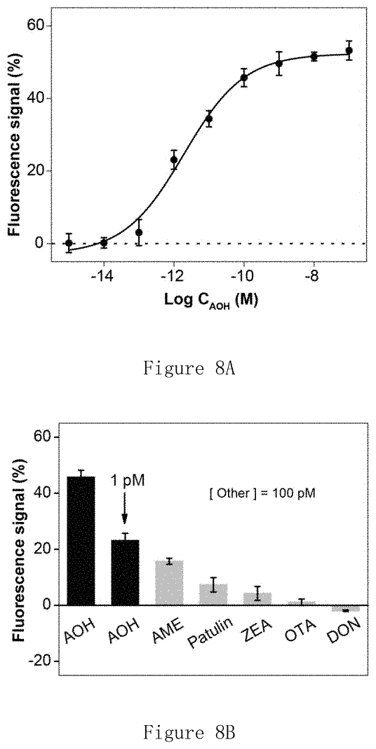

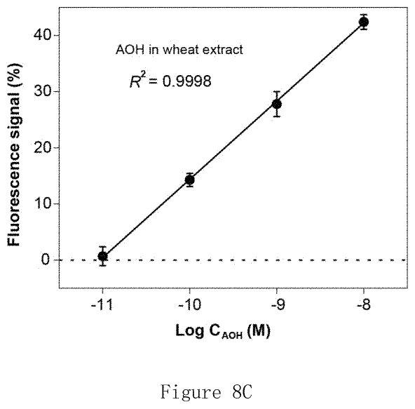

The result is as shown in FIG. 8A. The detection limitation is 666 fM, and the dynamic range is 666 fM-10 nM. As shown in FIG. 8B, the fluorescent signal of 1 pM AOH decreased much than that of 100 pM other toxin small molecules, thus the he sensor has ultrahigh target selectivity (>100). As shown in FIG. 8C, after the wheat extract containing AOH was diluted for 100 times, and detected directly without any sample pretreatment, the detection limitation is 100 pM, and the linear dynamic range is 10 pM-10 nM (R2=0.9998), in which the detection limitation is much less than 10 ng/g (6 nM) of the State standard limitation of AOH in the wheat.

### Embodiment 8. SPME-OWS has Advantages that it is Conveniently Controlling of the Dynamic Interval

SPMES-OWS of the invention realized conveniently controlling of the detection dynamic interval. Hydroxylation, silylanization, coupling, sealing and restoring process of the optic fiber surface are same as Embodiment 1. Taking the detection of Kana A as one example, the method of the invention can conveniently control of the detection dynamic interval by change components of the buffer. Taking the detection of SDM as one example, the method of the invention can conveniently control of the detection dynamic interval by change SPME layers. All experiment processes are same as Embodiments 4-7, only requiring changes of the buffer and SPME layers.

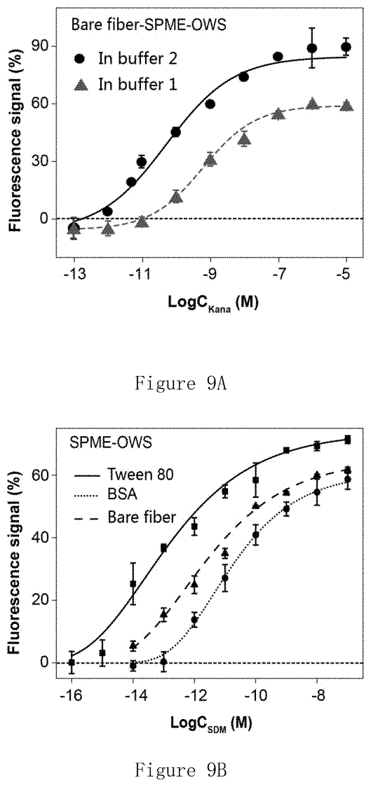

The result is as shown in FIG. 9A. Detecting Kana A using SPME-OWS without sealing layer, when using buffer 2, the detection dynamic range is 10-12 to 10-7 M; when using buffer 1, the detection dynamic range is 10-10 to 10-7M. Components of the buffer 1 and buffer 2 are substantially same, only the buffer 2 containing trihydroxymethylaminomethane, 20 mM, and the buffer 1 containing phosphate, 10 mM. Trihydroxymethylaminomethane also has OH group and multiple amino-groups, which can be competitively extracted by the fiber surface and decrease enrichment of Kana A by SPME. Therefore, the detection sensitivity is low, and the dynamic range is narrow when using the buffer 1.

As shown in FIG. 9B, the detection dynamic interval can be controlled of the detection dynamic interval by change SPME layers for detection of SDM. For example, when using the sealing layer of Tween 80, the detection dynamic range is 10-16 to 10-7 M; the detection dynamic range is 10-15 to 10-7M without the sealing layer, when using the sealing layer of BSA, the detection dynamic range is 10-13 to 10-7 M.

### Embodiment 9. SPMES-OWS has the Superior Interface Regeneration Performance

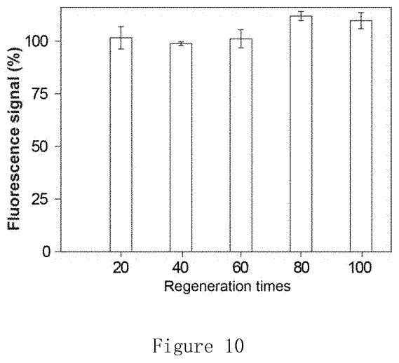

The fiber surface of SPMES-OWS of the invention has the superior interface regeneration performance. As shown in FIG. 10, taking SPME-OWS sealed with Tween 80 for the detection of DEHP of the invention as one example, after the interface was regenerated on the fiber surface for 100 times, change of the fluorescent signal hybridized with cDNA is ±6%, which is very stable.

It should be understood that the foregoing discussion, embodiments and examples merely present a detailed description of certain preferred embodiments. It will be apparent to those of ordinary skill in the art that various modifications and equivalents can be made without departing from the spirit and scope of the invention.

1-12. (canceled)

13. A fiber-optic wave guide sensor of aptamers having functions of in situ target enrichment and purification, combining SPME and aptamers, synchronously assembling extraction layer SPME having high efficiency target extraction capability and aptamers having target specificity on fiber-optic sensing interface, and said extraction layer SPME being a bare fiber or Tween 80.

14. The fiber-optic wave guide sensor of aptamers of claim 1, wherein said aptamers having target specificity being NH₂-(EG)₁₈-TGGGGGTTGAGGCTAAGCCGAGTCACTAT (SEQ ID NO: 1), or NH₂-(EG)₁₈-GAGGGCAACGAGTG TTTATAGA (SEQ ID NO: 2), or NH₂-(EG)₁₈-CTTTCTGTCCTTCCGTCACATCCCACGCATTCTCCACAT (SEQ ID NO: 3), or NH₂-AAAAAAAAAATAGCTTAACTAGTGTTCAAGCTG (SEQ ID NO: 12), said aptamers having target specificity being tethered on the fiber surface.

15. A method for detection of small molecules, wherein the method realizes quantitative detection of small molecules targets based on that small molecules targets and the aptamers complementary short strand DNA (cDNA) competitively bind with aptamers tethered on the fiber surface, combining SPME and aptamers, synchronously assembling extraction layer SPME having high efficiency target extraction capability and aptamers having target specificity on the fiber-optic sensing interface, and said extraction layer SPME being the bare fiber or Tween 80.

16. The method of claim 15, wherein the SPME on the fiber surface high effectively enriched small molecules in the solution nearby the fiber surface, which substantially bind small molecules with aptamers tethered on the fiber surface.

17. The method of claim 16 comprising the following steps:
step 1) hydroxylation of the optic fiber surface;
step 2) silylanization of the optic fiber surface;
step 3) aptamers coupling of the optic fiber surface; and
step 4) restoring and sealing.

18. The method of claim 17, wherein step 1) hydroxylation of the optic fiber surface: firstly, the optical fiber with clean surface is dipped into a 3:1 v/v concentrated sulfuric acid and a 30% hydrogen peroxide mixing solution at 100-120° C. for 1 h, then, the fiber is taken from the mixing solution and washed to neutral with the ultrapure water, followed by blowing dry with nitrogen and drying in an oven at 70-90° C. for 4-6 h, taking the fiber in the dryer and cooling to room temperature.

19. The method of claim 17, wherein step 2) silylanization of the optic fiber surface: the above fiber is immersed APTS anhydrous toluene solution at room temperature for 1-2 h, followed by rinsing with Anhydrous toluene, toluene-ethyl alcohol (v/v=1:1) and ethyl alcohol wash (three time), blowing dry with nitrogen and drying in an oven at 180° C. for 4-6 h, taking the fiber in the dryer and cooling to room temperature.

20. The method of claim 17, wherein step 3) aptamers coupling of the optic fiber surface: the optical fiber of silylanization is immersed in 10 mM phosphate buffered solution (PB) containing glutaraldehyde for 4 h at room temperature, after the reaction being finished, washed with the ultrapure water three times, blowing dry with nitrogen, the fiber is then immersed in the amino modified aptamers solution 6-8 h at room temperature, washed then with the ultrapure water three times.

21. The method of claim 17, wherein step 4) restoring and sealing: the above fiber is immersed in sodium borohydride (NaBH₄) solution for 30 minutes, sealing the fiber interface with a certain concentration of extractant, for example, Tween 80 solution (when fabricating SPME-OWS of the bare fiber, the fiber interface is sealed without the extractant), washed then with the ultrapure water three times and stored in the refrigerator of 4° C.

22. The method of claim 17 further comprising the following steps:
step 5) the optic fiber is assembled into the reaction chamber of the waveguide sensor, after the baseline being stabled, pumping the mixed solution containing a certain concentration of small molecule target and complementary chains of fluorescent modified aptamers in the reaction chamber, measuring the change of fluorescence signal in real time;
step 6) the fiber is flushed with solution of sodium dodecyl sulfate (SDS) to regenerate the sensor interface; repeating step 5);
step 7) drawing the working curves of optical waveguide sensor detecting different targets; and
step 8) selectivity test: targets of step 5) are changed to substances of selectivity test.


SEQUENCE LISTING (12 sequences)

SEQ ID NO: 1 - DNA, 29 bp, Artificial Sequence
  Synthetic NH2-Kana
  modified_base (1)..(1): NH2-(EG)18
tgggggttgaggctaagccgagtcactat

SEQ ID NO: 2 - DNA, 22 bp, Artificial Sequence
  Synthetic NH2-SDM
  modified_base (1)..(1): NH2-(EG)18
gagggcaacgagtgtttataga

SEQ ID NO: 3 - DNA, 39 bp, Artificial Sequence
  Synthetic NH2-PAE
  modified_base (1)..(1): NH2-(EG)18
ctttctgtccttccgtcacatcccacgcattctccacat

SEQ ID NO: 4 - DNA, 27 bp, Artificial Sequence
  Synthetic NH2-HSA
  modified_base (1)..(1): NH2
aaaaaaaaaagtgccgaaatacggcac

SEQ ID NO: 5 - DNA, 12 bp, Artificial Sequence
  Synthetic c-Kana-Cy 5.5
  modified_base (12)..(12): Cy 5.5
atagtgactcgg

SEQ ID NO: 6 - DNA, 14 bp, Artificial Sequence
  Synthetic c-SDM-Cy 5.5
  modified_base (14)..(14): Cy 5.5
aaacactcgttgcc

SEQ ID NO: 7 - DNA, 14 bp, Artificial Sequence
  Synthetic c-PAE-Cy 5.5
  modified_base (14)..(14): Cy 5.5
ggatgtgacggaag

SEQ ID NO: 8 - DNA, 32 bp, Artificial Sequence
  Synthetic c-HSA-Cy 5.5
  modified_base (32)..(32): Cy 5.5
agcttatgcgtagcctctagtgattaacgcag

SEQ ID NO: 9 - DNA, 39 bp, Artificial Sequence; identical to SEQ ID NO: 3
  Synthetic Cy 5.5-PAE
  modified_base (1)..(1): Cy 5.5

SEQ ID NO: 10 - DNA, 29 bp, Artificial Sequence
  Synthetic Cy 5.5-Kana
  modified_base (1)..(1): Cy 5.5
tgggggttgaggctaagccgagtcactat

SEQ ID NO: 11 - DNA, 22 bp, Artificial Sequence
  Synthetic Cy 5.5-SDM
  modified_base (1)..(1): Cy 5.5
gagggcaacgagtgtttataga

SEQ ID NO: 12 - DNA, 33 bp, Artificial Sequence
  Synthetic aptamer
  modified_base (1)..(1): NH2
aaaaaaaaaatagcttaactagtgttcaagctg